(19) United States
(12) Patent Application Publication
(10) Pub. No.: US 2019/0008726 A1
(43) Pub. Date: Jan. 10, 2019

(54) System And Method For Saliva Replenishment And Control

(71) Applicant: Jennifer Rebecca ALTOUNIAN, White Lake, MI (US)

(72) Inventor: Jennifer Rebecca ALTOUNIAN, White Lake, MI (US)

(21) Appl. No.: 16/052,860

(22) Filed: Aug. 2, 2018

Related U.S. Application Data
(63) Continuation-in-part of application No. 15/373,555, filed on Dec. 9, 2016.
(60) Provisional application No. 62/265,172, filed on Dec. 9, 2015.

(51) Int. Cl. A61J 7/00; A61M 31/00; A61M 1/00; A61C 19/06; A61M 3/02

(52) U.S. Cl. CPC A61J 7/0053; A61M 31/00; A61M 1/0058; A61M 2210/0631; A61M 3/02; A61M 2202/0466; A61M 2210/0625; A61C 19/063

(57) ABSTRACT

Disclosed is a system for providing artificial saliva. The system can include the mouthpiece, a portable supply unit, and a stationary supply unit. The portable supply unit can be adapted to be carried by a user. The portable supply unit can include a first fluid system, a first interface module, and a first control module. The first fluid system can be adapted to fluidly couple to the mouthpiece.

### CROSS-REFERENCE TO RELATED APPLICATIONS

This application is a continuation-in-part of U.S. patent application Ser. No. 15/373,555 filed on Dec. 9, 2016; which claims the benefit of U.S. Provisional Application No. 62/265,172, filed on Dec. 9, 2015. The entire disclosure of the above application is incorporated herein by reference.

### FIELD

The present disclosure relates to systems and methods for moisture control and, more particularly, to mouthpieces and fluid delivery and removal systems.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Saliva production and removal is important to several biological functions including swallowing, digestion, and oral hygiene. For example, enzymes in saliva aid digestion. As another example, saliva hydrates the mouth and throat, aiding swallowing and promoting oral hygiene. Salivary glands may become impaired and/or damaged due to accident, trauma, illness, disease, and/or medical treatments including surgery, medications, radiation therapy, and chemotherapy treatments. In some cases, the accident, trauma, illness, disease, or medical treatment can seriously impair or permanently damage the salivary glands and, as a consequence, render normal salivary production impossible.

As one example, a condition referred to as Xerostomia in the medical community afflicts millions of people worldwide. Xerostomia is used to refer to a condition in which saliva production is impaired or absent. Xerostomia can be caused by various diseases such as Sjögren's syndrome, human immunodeficiency virus (HIV), Alzheimer's disease, diabetes, cystic fibrosis, lupus, and rheumatoid arthritis. As another example, people undergoing cancer treatments such as radiation therapy and chemotherapy to the head and neck often experience a loss in saliva production, and the loss can be permanent. Medications may also diminish saliva production. Saliva production may also diminish as an individual ages and can become problematic at advanced ages.

Several problems can arise in an individual whose saliva production or removal is compromised. Without the ability to produce or remove saliva, an individual may not properly produce the enzymes necessary to properly digest food. Tooth decay, painful sores in the mouth, problems swallowing, and the inability to eat and speak can arise and can cause other significant health issues. For example, psychological stress and/or other problems can develop. At a minimum, an individual's quality of life can be negatively impacted.

Alternatively, saliva production may be increased and/or swallowing inhibiting conditions may exist. These conditions may lead to a buildup of saliva in a mouth of a patient. Further, during selected procedures a fluid removal from the oral cavity may be selected. Accordingly, it may be selected to assist in removal of fluid, such as saliva, from a mouth of a subject.

Accordingly, a device, which can be used to replenish and remove saliva and control saliva production, as well as provide a more convenient way to deliver medications to patients is desired. Several devices have been developed. However, these devices can be improved upon. Specifically, the comfort, versatility, operation, and appearance can be improved upon. Through such improvements, patient compliance with therapies directed to saliva production and other health issues can be improved along with the patient's quality of life. Additionally, such improvements can make certain treatments such as cancer treatments more tolerable and provide for a more stable overall health.

### SUMMARY

In one form, the present disclosure provides a mouthpiece. The mouthpiece can include a hydrophilic foam surrounded by a heat shrinkable layer. The mouthpiece may further includes, or alternatively to being surrounded by a heat shrinkable piece, the hydrophilic foam may be connected to a tube member. In various embodiments, the mouthpiece includes only the hydrophilic foam without a co-extensive over-layer or surrounding layer. The foam may be connected to a tube. The hydrophilic foam member can be adapted to engage a lower dental arch adjacent a first tooth on a first side of a mouth, within the dental arch, adjacent a cheek, between an upper lip and an upper dental arch, or other appropriate areas within the mouth. The hydrophilic foam member can include a first passage extending between a vestibule on the first side of the mouth. Optionally, an anchoring member can be adapted to engage the lower dental arch or a patient's lip. The first tube can extend within the mouth cavity proper from the hydrophilic foam member adjacent to an inner gum line. The second tube can extend within the vestibule on the first side of the mouth adjacent to an outer gum line. The second tube can include a first end fluidly coupled to the first passage and a second end extending outside of the mouth.

The present disclosure also provides a system that can include the mouthpiece, a portable supply or removal unit, and a stationary supply unit. The portable supply or removal unit can be adapted to be carried by a user. The portable supply or removal unit can include a first fluid system, a first interface module, and a first control module. The first fluid system can be adapted to fluidly couple to the mouthpiece. The first fluid system can supply or remove saliva replenishment fluid to the mouth via the mouthpiece and/or remove fluid from the mouth via the mouthpiece based on first operational settings. The first interface module can receive a first input and can communicate the first operational settings with the stationary supply unit. The first control module can selectively adjust the first operational settings based on the first input and second operational settings communicated by the stationary supply unit. The stationary supply unit can include a second fluid system, a second interface module, and a second control module. The second fluid system can be adapted to fluidly couple to the mouthpiece. The second fluid system can supply saliva replenishment fluid to the mouth via the mouthpiece and/or remove fluid from the mouth via the mouthpiece based on the second operational settings. The second interface module can receive a second input and can communicate the second operational settings with the portable supply unit. The second control module can selectively adjust the second operational settings based on the second input and the first operational settings.

In another form, the present disclosure provides methods related to a mouthpiece and a system according to the present disclosure.

Further areas of applicability of the present disclosure will become apparent from the detailed description provided hereinafter. It should be understood that the detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIGS. 1a and 1b are an environmental perspective view a first exemplary mouthpiece according to the present disclosure;

FIG. 2 is an environmental perspective view of the mouthpiece shown in FIGS. 1a and 1b;

FIG. 3 is an exploded view of the mouthpiece shown in FIG. 2;

FIGS. 4a and 4b are illustrating an exemplary tube used in the mouthpiece shown in FIGS. 1a-3;

FIG. 5 is an optional stiffening member used in the mouthpiece shown in FIGS. 1a-3;

FIGS. 6a-c illustrates a tube associated with the mouthpiece, according to various embodiments;

FIGS. 7a-7d represent an alternate upper and lower saliva replenishment prosthesis according to the present teachings;

FIG. 8 represents the installation of a lower mouth prosthetic according to the present teachings;

FIGS. 9a-9c represent an alternate upper saliva replenishment prosthesis according to the present teachings;

FIGS. 10a-10d represent an alternate upper saliva replenishment prosthesis according to the present teachings;

FIGS. 11a-11k represent an alternate saliva replenishment prosthesis according to the present teachings;

FIGS. 12a-12f represent over the tooth saliva replenishment prosthesis according to the present teachings;

FIGS. 13a-13d represent alternate saliva replenishment prosthesis according to the present teachings;

FIG. 14 illustrates a tube associated with the mouthpiece, according to various embodiments;

FIG. 14A and FIG. 14B are cross-sectional views of a capture portion of the mouthpiece of FIG. 14;

FIGS. 15A-15B illustrate a mouthpiece assembly with the tube illustrated in FIG. 6A-6C;

FIG. 16 illustrates a mouthpiece with the tube illustrated in FIG. 14;

FIG. 17 is an environmental view of a mouthpiece in a mouth, according to various embodiments;

FIG. 18 is a detail cross-sectional view of a mouthpiece, according to various embodiments, with a connection member;

FIG. 19 is a detail cross-sectional view of a mouthpiece, according to various embodiments, with a connection member; and

FIG. 19A is a detail cross-sectional view of a connection member and tube connection for a mouthpiece.

### DETAILED DESCRIPTION

The following description is merely illustrative in nature and is in no way intended to limit the disclosure, its application, or uses. For purposes of clarity, the same reference numbers will be used in the drawings to identify similar elements. As used herein, the phrase at least one of A, B, and C should be construed to mean a logical (A or B or C), using a non-exclusive logical or. It should be understood that steps within a method may be executed in different order without altering the principles of the present disclosure.

The present disclosure provides a mouthpiece that can be placed, and in various embodiments attached, to a lower portion of a mouth and can be used to replenish or remove and/or control saliva in a patient or user. The mouthpiece may further be placed in an upper portion of a mouth and/or both an upper and lower portion of the mouth. The user can be a male or female and can be an adult or child. The mouthpiece can be used during treatment of temporary conditions such as a temporary loss of swallowing capability due to a stroke or trauma, or chronic conditions and diseases such as Xerostomia and cancers affecting salivary function. The mouthpiece can be used to supply and/or remove fluid from the mouth. The mouthpiece is designed so that it can be relatively inconspicuous and minimally intrusive, and can be continuously worn for prolonged periods, (e.g. about 2 hours to about 8 hours, including about 6 hours) for example days and weeks. The mouthpiece is further designed so that it can be used to supply a saliva replenishment fluid in a manner that mimics a normal saliva flow within the mouth. By incorporating such design features, the mouthpiece can be comfortably used without compromising chewing, eating, speaking, and sleeping, and can promote patient compliance with therapies dependent on the use of the mouthpiece.

In various aspects, the mouthpiece can be individually sized and made to fit a particular user. The mouthpiece can have a modular construction that further enables components of the mouthpiece as produced to be altered and custom fit to a particular patient. The modular construction can also enable one or more components of the mouthpiece to be individually replaced without the need for another complete replacement mouthpiece. The components can be individually replaced to maintain a desired sanitary condition of the mouthpiece.

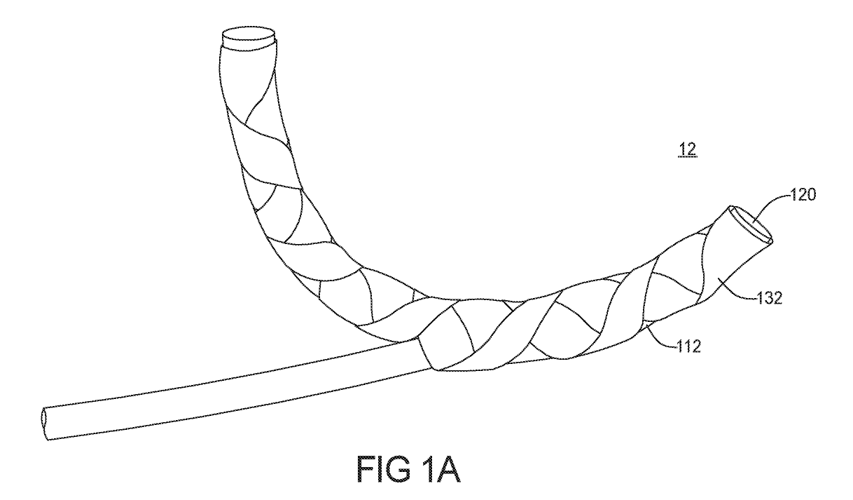

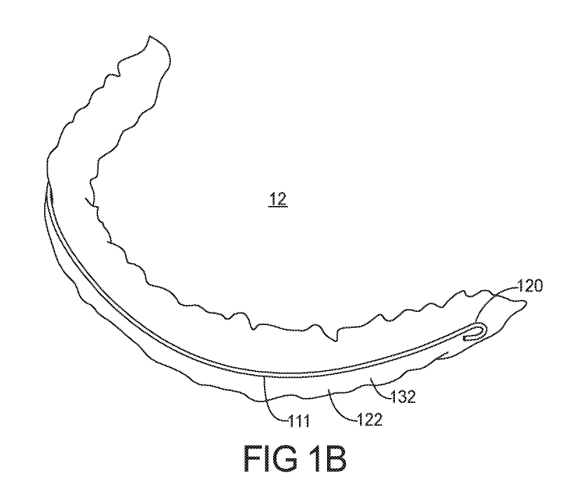

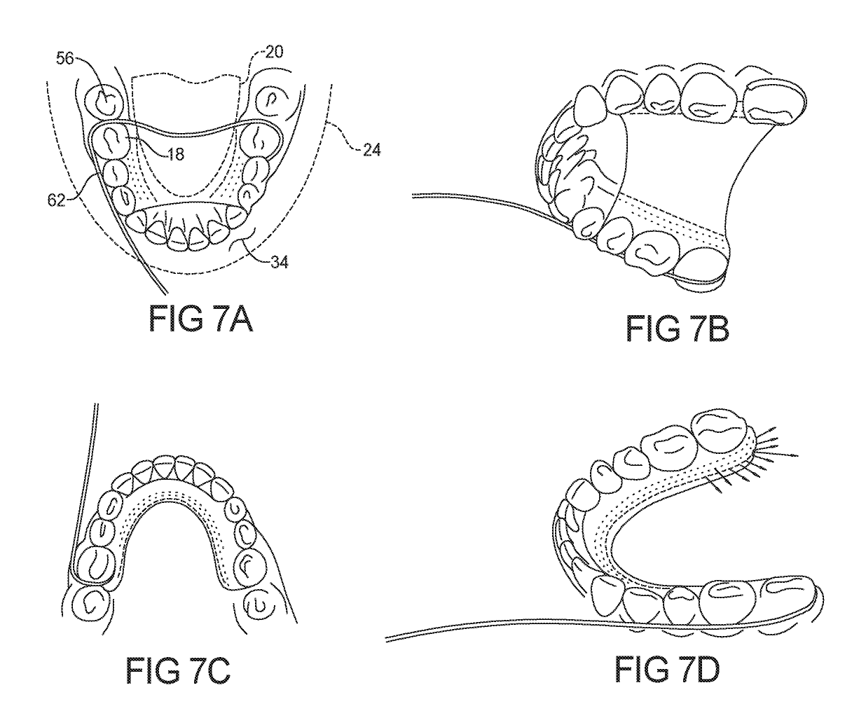

With particular reference to FIGS. 1A-3, environmental views of a lower portion of an exemplary mouthpiece 12 according to the present disclosure. The mouthpiece 12 can be anchored adjacent the lower teeth 20 anchored in a lower jaw bone by gingiva or gum. The lower dental arch includes alveolar processes that receive roots of the lower teeth, the lower teeth, and portions of the gum covering the alveolar processes and surrounding the lower teeth. The lower teeth generally include about 12 deciduous teeth in a child and about 16 permanent teeth in an adult. For exemplary purposes, as illustrated in FIG. 7A, the lower teeth presented include 14 teeth: four incisors, two canines, four premolars, and four molars. The lower teeth include lingual surfaces 18 facing the mouth cavity proper and a tongue 20 (shown in phantom), labial or buccal surfaces 62 facing the vestibule 34 and a cheek 24 (shown in phantom) and lips (not shown), and surfaces of contact between adjoining teeth. The lower teeth further include crowns facing upper teeth of an upper jaw bone and defining a lower bite surface or plane. Together, the lower teeth and the gum define an inner gum line and an outer gum line. Openings of a submandibular duct within the mouth cavity proper, which may be referred to as the Wharton's duct, are illustrated by openings.

The mouthpiece 12 can include a first foam anchoring member 100, a second member 102 annularly disposed about the first foam anchoring member 100, a first tubular member 104 partially disposed within the first foam anchoring member 100 and supported by the second member 102. The first and second anchoring members 100 and 102 can be sized to fit within the spaces between the lower lip and the lower teeth, with the first tubular member protruding from the mouth to provide vacuum or saliva substitute to the mouth. Optionally, the mouthpiece can include a deformable first foam anchoring member 100 can be generally solid structures and can have various shapes adapted to fit within the spaces adjacent to the molars and adjoining teeth of the lower teeth.

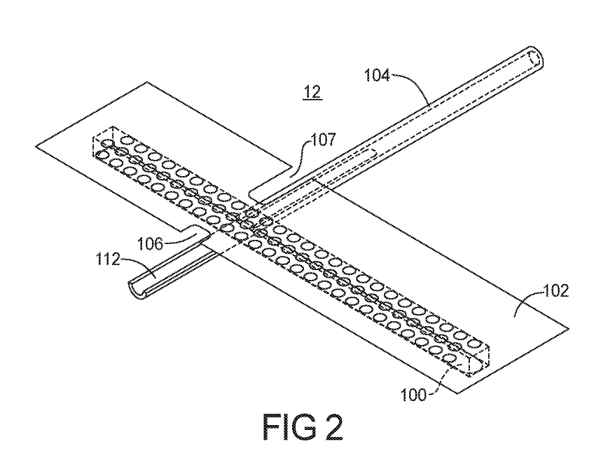

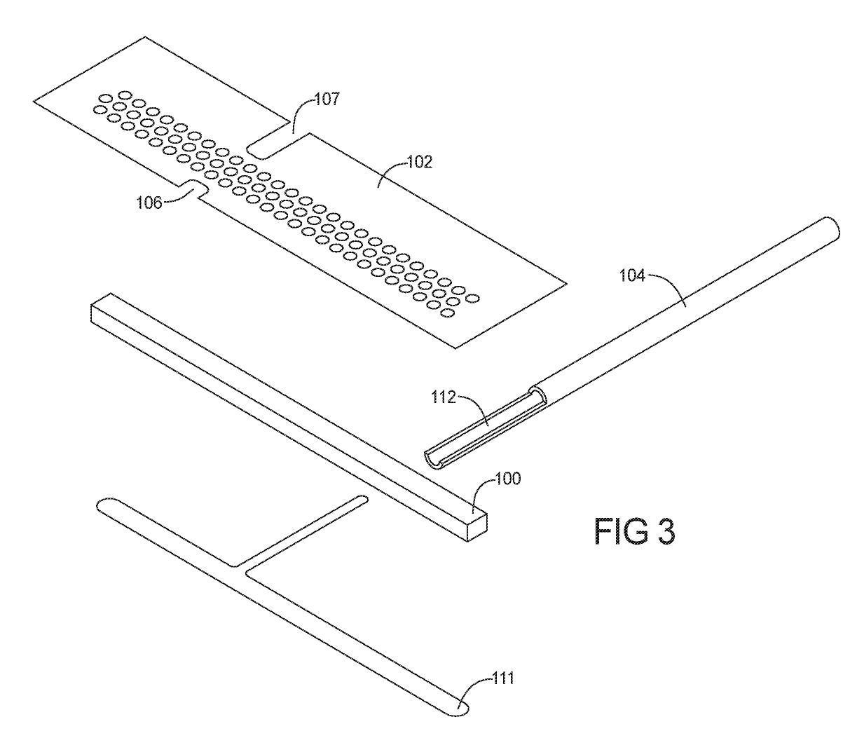

With additional reference to FIGS. 2 and 3, the mouth piece 12 can include a foam member 120, an anchoring tube 122 (shown here flattened), and the first tubular member 104. The hydrophilic foam member 120 extends about the lower gum. The hydrophilic foam member 120 can have a generally tapered polyhedral shape as illustrated, or can have a contoured shape resembling a natural tooth that may otherwise reside in the space.

The foam member may be any appropriate foam material. In various embodiments, the foam member 100 is an open cell foam. The foam member 100 may be Hydrasorb® Foam Hydrasorb® the name of a group of medical-grade polyurethane, hydrophilic foams. This foam is manufactured from a base material of polyether polyisocynate resins. Hydrasorb® is sterilizable and/or sterilized and can be Die cut from 18″×36″ sheets of a selected thickness, such as about 1 centimeter (cm) to about 3 cm or molded to a selected shape. The foam member 100 may have selected properties, such as Hydrophilic Absorption Capacity (Water): up to 15× Dry Wt. [ASTM D1667]; Cell Structure (DRY Avg.): 86 Cells/Linear In; Density (Nominal/DRY): 7.5 lb/ft3 [ASTM D3574]; Elongation % (DRY Avg.): 650% [ASTM D3574]; Expansion in Water (length)(Avg.): 31% (Avg.) [ASTM F1087]; Foam Moisture Content (DRY Avg.): 3.56% [Karl Fischer Method]; Indention Force Deflection (IDF): [ASTM D3574 Test B1] IDF @ 25%: (N) 133% IDF @ 65%: (N) 346%; Resiliency/Rebound Test (RT [ASTM D3574 Test B1] RT @ 25%: (N) 121%; Compression Set (DRY Avg.): [ASTM D3574] 25%: 16.0% 50%: 36.0%; and Tensile Strength (Dry): 30.0 lbf/In2 [ASTM D3574 Test B1].

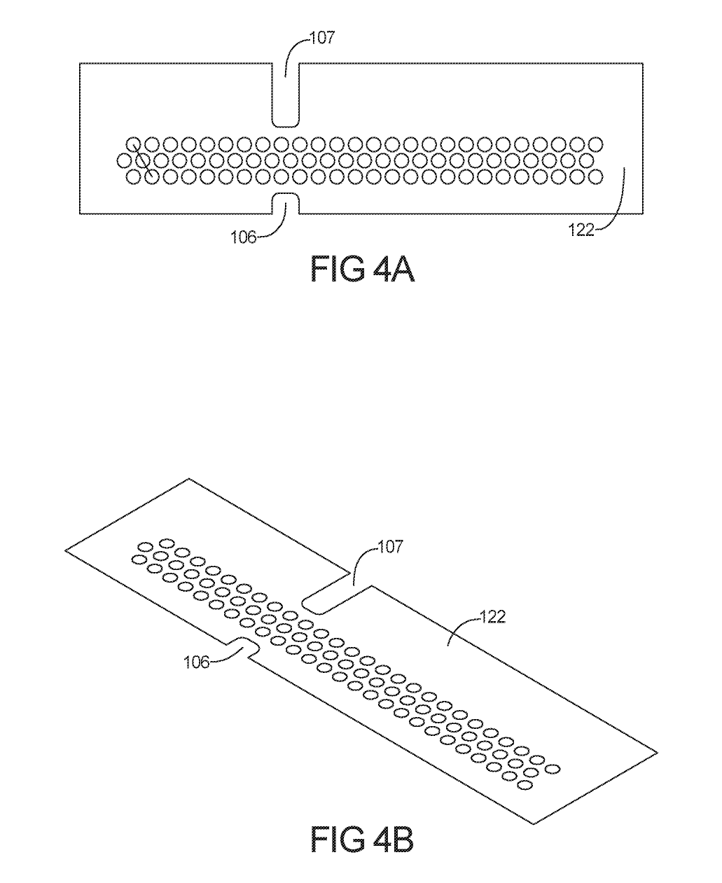

As shown in FIGS. 4A and 4B, the anchoring tube 122 is preferably perforated two accept saliva from the mouth or saliva replacement tube 104. The size and shapes of these perforations can vary. In a flattened configuration, a notch or pair of notches 106, 107 are utilized to be positioned about the tube 104 and the hydrophilic foam 102.

The front surface 132 can be adapted and disposed to engage one or more of the surface of contact, the lingual surface, and the buccal surface of the molar. The front surface 132 can be further disposed to allow one end of the tube 104 to exit the front surface 132 adjacent the lingual surface of the molar and an opposite end of the passage to exit the front surface adjacent the buccal surface of the molar. In this way, the front surface 132 can be disposed to allow the first tubular member 104 to extend from the front surface 132 adjacent the lingual surface of the molar, and the second tubular member to extend from the front surface adjacent the buccal surface 62 of the molar. The front surface 132 can be generally flat as illustrated by the present example and, optionally, can include a portion complementary to the adjoining surface of contact of the molar. In this way, the front surface can engage and thereby resist relative movement between the first foam anchoring member 100 and the molar 56.

In various aspects, the first and second anchoring members 100 and 102 can be made in a mirror image to that described above. In this way, the mouthpiece 12 may be configured so that the tubular member 104 exits the mouth 10 on the left side of the user.

In various aspects, the first and second anchoring members 100 and 102 can be attached in any suitable manner. For example, a suitable adhesive such as an adhesive that adheres dentures to a gum may be used. In various aspects, the first and second anchoring members 100 and 102 can be attached in a semi-permanent manner using a bone fastener. In various aspects, the first and second anchoring members 100 and 102 can be made from any suitable dental material which allows saliva infiltration. Suitable dental materials include, but are not limited to, biocompatible polymers such as acrylic materials, and metals such as titanium.

FIG. 5 represents a metal deformable support member 111 which can be inserted adjacent to the hydrophilic foam member 100, as illustrated in FIGS. 1B and 3. This metal deformable support member can then be used to form the mouthpiece prior to insertion between the lip and gum adjacent to the lower teeth.

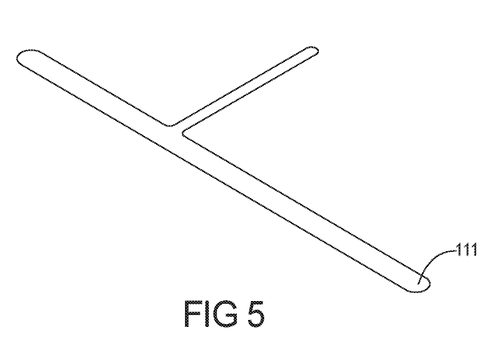

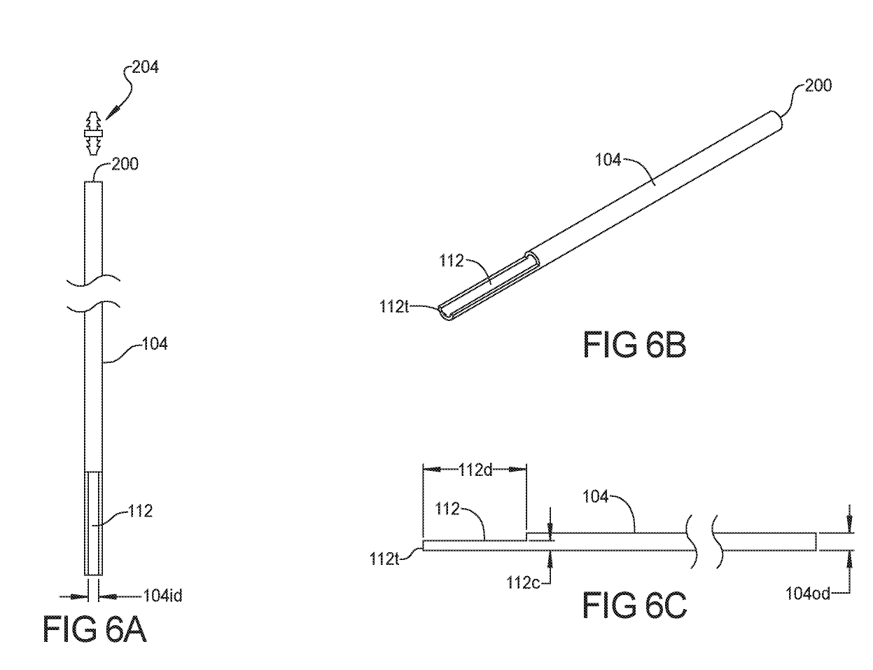

FIGS. 6A-6C represent the tube 104 which is placed adjacent to the hydrophilic foam member and within the heat shrink material of tube 102. As described above, a portion of the tube is passed though the slots 106 and 107. The tube 104 has a first portion which is perforated or notched 112 to allow suction to be applied to the open pore hydrophilic foam 100.

FIGS. 7A-7D represent an alternate upper and lower saliva replenishment prosthesis according to the present teachings. The upper and lower prosthesis are made of materials having a soft durometer. They define an inner passage and a plurality of apertures which link an outer surface of the prostheses with the inner aperture. As can be seen, each prosthesis utilizes an outer supply or extraction tube which can be coupled to a fluid supply or a vacuum as described above.

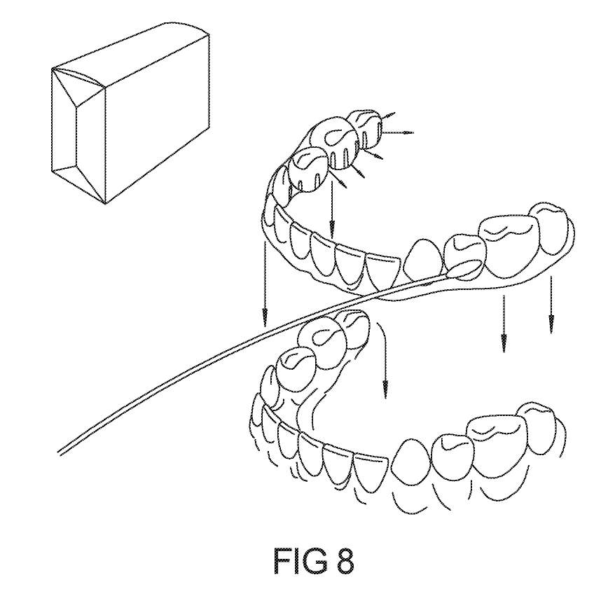

FIG. 8 represents the installation of a lower mouth prosthetic according to the present teachings. Shown is a film layer, which is coupled to the teeth using a water soluble adhesive. The fluid extraction tube is placed along the outside of the tooth ridge. Should a vacuum be drawn through the tube, fluid is drawn through apertures defined through the polymer layer. This configuration can be used as both the upper and lower prosthesis are made of materials having a soft durometer. They define an inner passages between the teeth which can us used to draw out saliva. The plurality of apertures link an outer surface of the prostheses with the inner aperture. As can be seen, each prosthesis utilizes an outer supply or extraction tube which can be coupled to a fluid supply or a vacuum as described above.

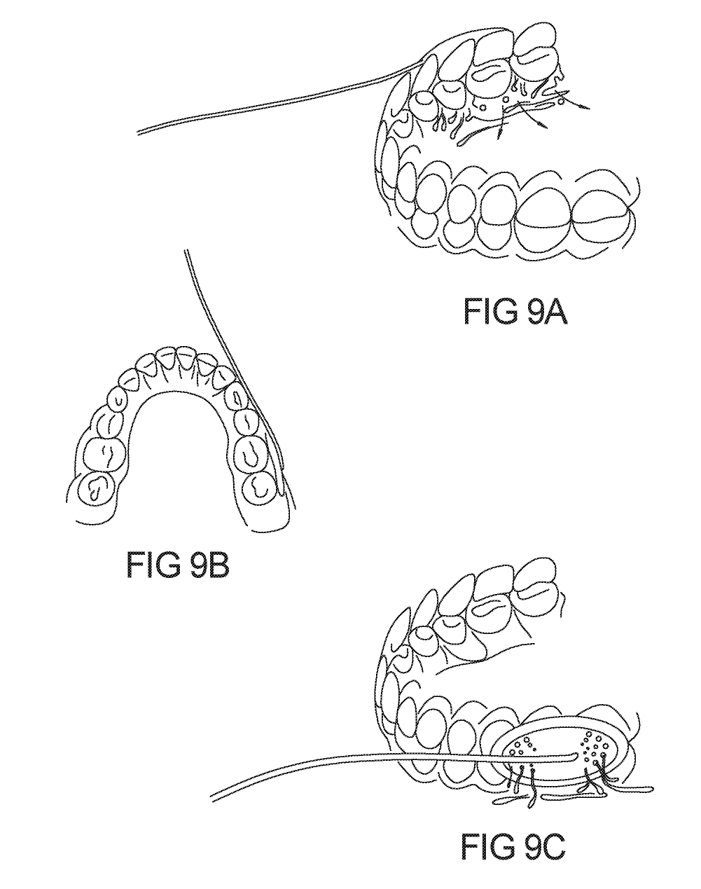

FIGS. 9A-9C represent an alternate upper saliva replenishment and/or removal prosthesis according to the present teachings. As is shown, a deformable polymer material has a fluid transport tube disposed therethrough. The deformable member is generally oval and is configured to be fixed to the molars between the tooth and the gum line. The device has a through passage and a plurality of excretion or vacuum holes. The fluid extraction tube is placed along the outside of the tooth ridge. Should a vacuum be drawn through the tube, fluid is drawn through apertures defined through the polymer layer. This configuration can be used as both the upper and lower prosthesis are made of materials having a soft durometer. They define an inner passages between the teeth which can us used to draw out saliva. The plurality of apertures link an outer surface of the prostheses with the inner aperture. As can be seen, each prosthesis utilizes an outer supply or extraction tube which can be coupled to a fluid supply or a vacuum as described above.

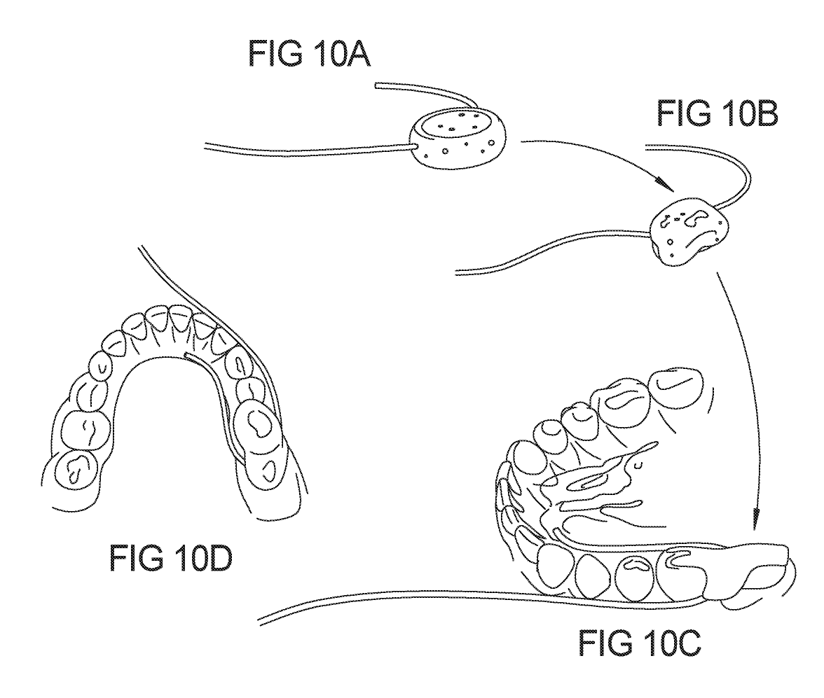

FIGS. 10A-10D represent an alternate saliva replenishment or removal prosthesis according to the present teachings. The fluid extraction tube is placed along the outside of the tooth ridge. The material that forms the saliva replenishment or removal prosthesis is plastically deformable and includes a plurality of through passages which remain open upon disposition over the teeth. The passages remain open because they have a surface which resists the sticking of one inner aperture surface to another. They are also configured to be strong enough not to collapse of the application of the vacuum. Should a vacuum be drawn through the tube, fluid is drawn through apertures defined through the polymer material. This configuration can be used as both the upper and lower prosthesis are made of materials having a soft durometer. They define an inner passages between the teeth which can us used to draw out saliva. The plurality of apertures link an outer surface of the prostheses with the inner aperture. As can be seen, each prosthesis utilizes an outer supply or extraction tube which can be coupled to a fluid supply or a vacuum as described above.

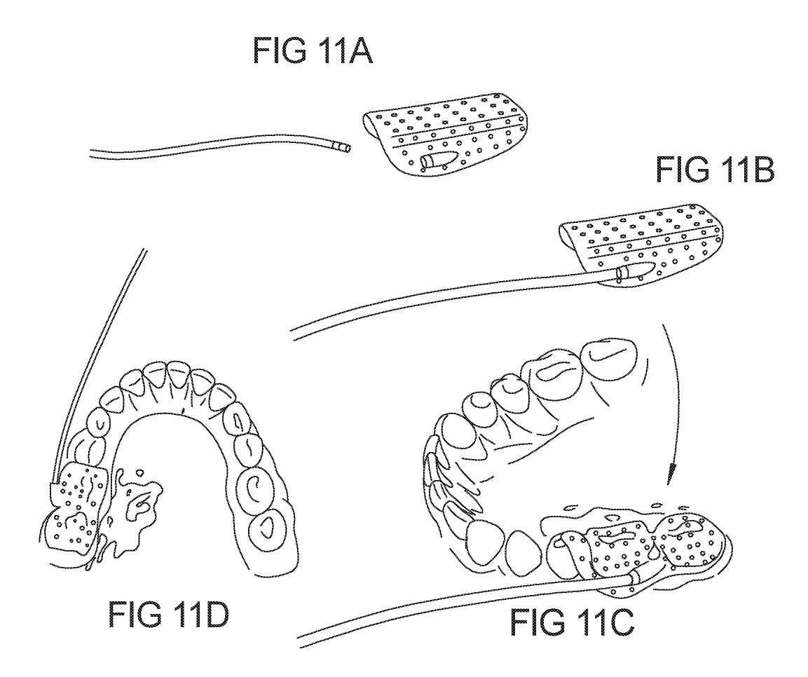

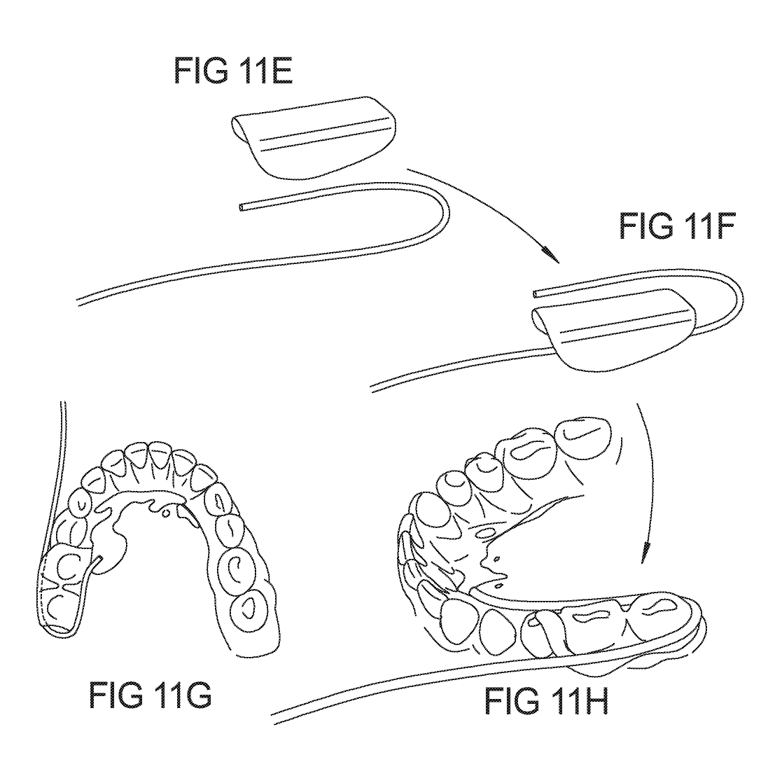

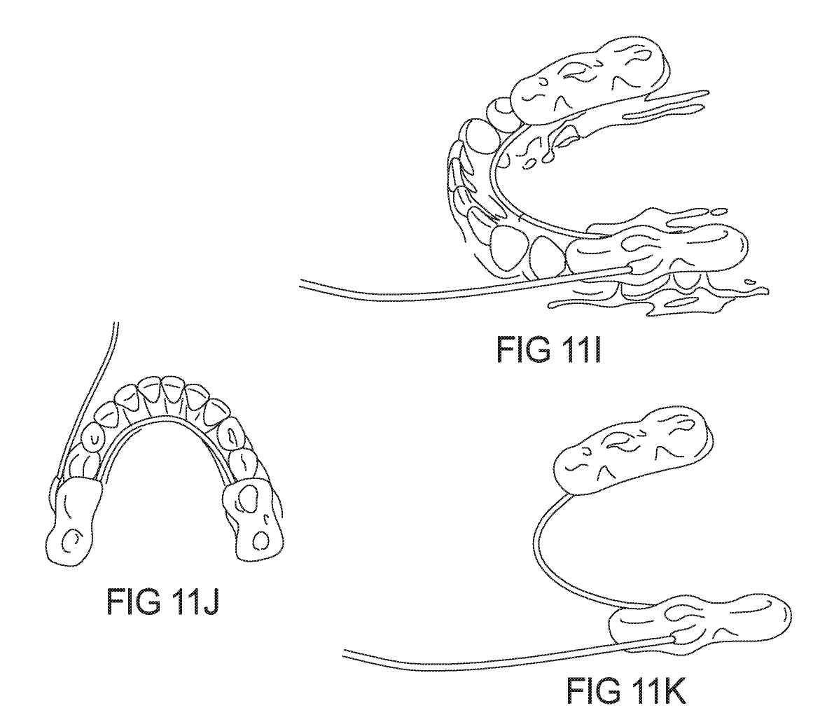

FIGS. 11A-11J represent an alternate saliva replenishment and/or removal prosthesis according to the present teachings. As is shown, a deformable polymer material has a fluid transport tube disposed therethrough. The deformable member is generally oval and is configured to be fixed to the molars between the tooth and the gum line. The device has a through passage and a plurality of excretion or vacuum holes. The fluid extraction tube is placed along the outside of the tooth ridge. Should a vacuum be drawn through the tube, fluid is drawn through apertures defined through the polymer layer. This configuration can be used as both the upper and lower prosthesis are made of materials having a soft durometer. They define an inner passages between the teeth which can us used to draw out saliva. The plurality of apertures link an outer surface of the prostheses with the inner aperture. As can be seen, each prosthesis utilizes an outer supply or extraction tube which can be coupled to a fluid supply or a vacuum as described above.

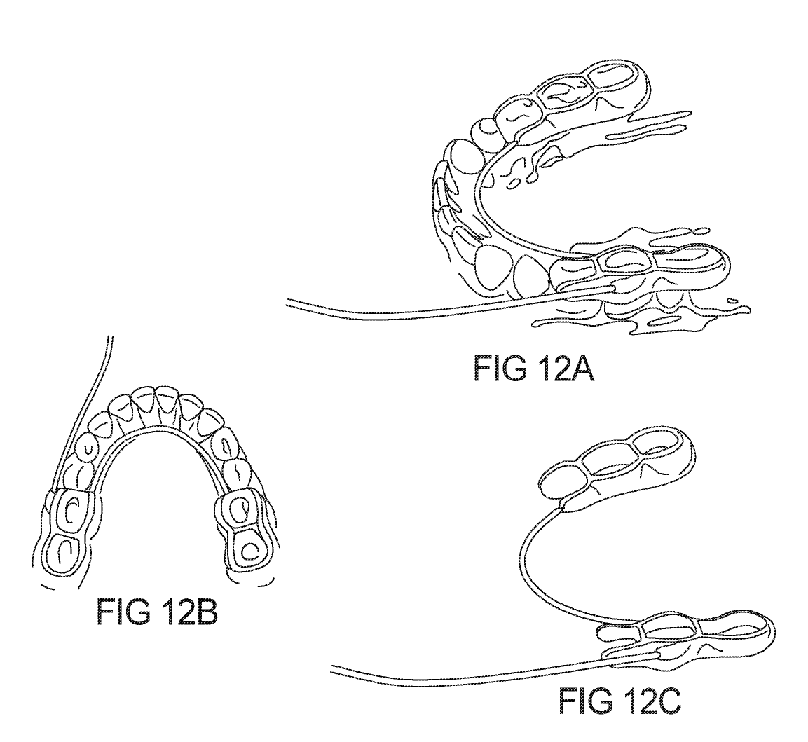

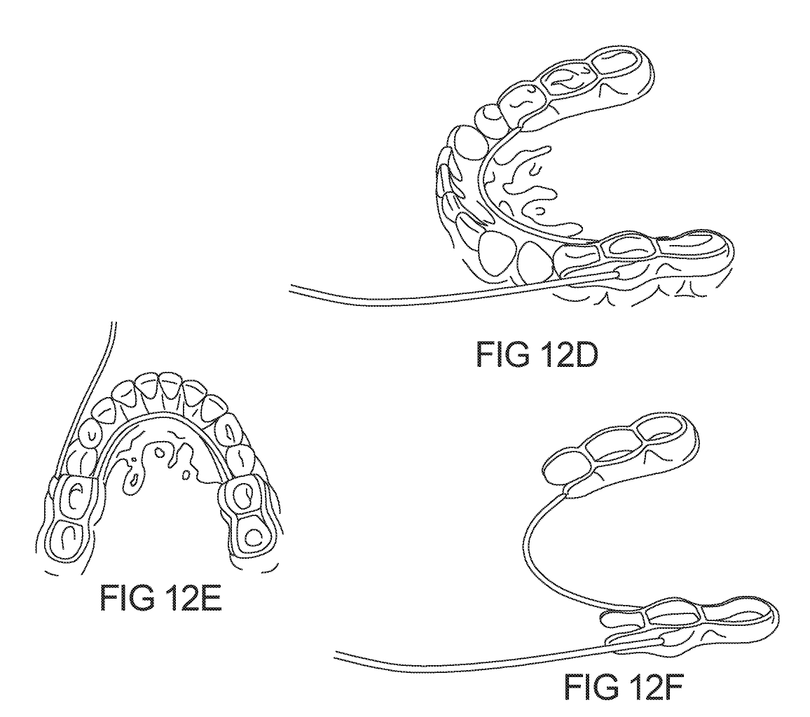

FIGS. 12A-12F represent an alternate saliva replenishment and/or removal prosthesis according to the present teachings. The prosthetic member is formed around the molars and do not interfere with the molar bite surface. Similar to the teachings in FIGS. 7A-7D, alternate upper and lower saliva replenishment and/or removal prosthesis according to the present teachings. As can be seen the prosthesis travels over the molar region and is positioned under the tongue at only a single location. The upper and lower prosthesis are made of materials having a soft durometer. They define an inner passage and a plurality of apertures which link an outer surface of the prostheses with the inner aperture. As can be seen, each prosthesis utilizes an outer supply or extraction tube which can be coupled to a fluid supply or a vacuum as described above.

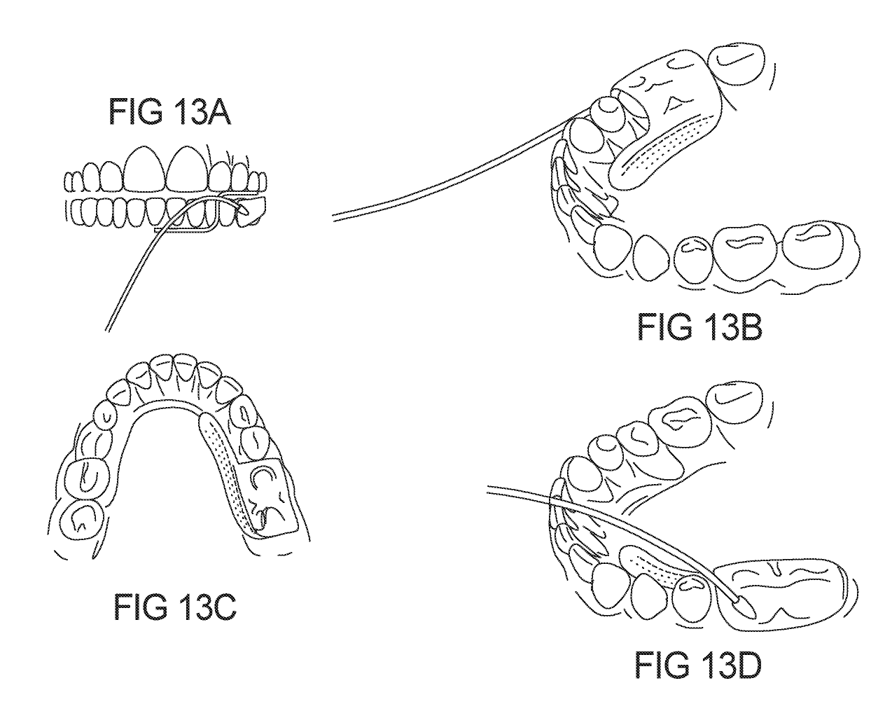

FIGS. 13A-13D represent an over the tooth saliva replenishment and/or removal prosthesis according to the present teachings. Similar to the teachings in FIGS. 7A-7D, alternate upper and lower saliva replenishment prosthesis according to the present teachings. As can be seen the prosthesis travels over the molar region and is positioned under the tongue at only a single location. The upper and lower prosthesis are made of materials having a soft durometer. They define an inner passage and a plurality of apertures which link an outer surface of the prostheses with the inner aperture. As can be seen, each prosthesis utilizes an outer supply or extraction tube which can be coupled to a fluid supply or a vacuum as described above.

In various aspects, the first and second anchoring members can be off-the-shelf components, semi-custom components, or custom components. As used herein, off-the-shelf components can refer to components made without features based on a particular user. Semi-custom components can refer to components made in advance that include a majority of predetermined features not based on a particular user and at least one feature based on a particular user. Custom components can refer to components specifically made for a particular user. The patient-specific features of a semi-custom component and a custom component can be formed based on a particular user's lower dental arch and surrounding mouth anatomy using various techniques such as dental impressioning techniques.

Each of the embodiments in FIGS. 7A-13D can utilize the foam member 100 which may be Hydrasorb® Foam Hydrasorb® the name of a group of medical-grade polyurethane, hydrophilic foams. This foam is manufactured from a base material of polyether polyisocynate resins. Hydrasorb®, as discussed above, and is sterilized and/or is sterilizable and can be Die cut from 18″×36″ sheets (⅛″ to ¾″ wetted thickness) or molded to a shape. Hydrophilic Absorption Capacity (Water): up to 15× Dry Wt. [ASTM D1667] Cell Structure (DRY Avg.): 86 Cells/Linear In. Density (Nominal/DRY): 7.5 lb/ft3 [ASTM D3574] Elongation % (DRY Avg.): 650% [ASTM D3574] Expansion in Water (length)(Avg.): 31% (Avg.) [ASTM F1087] Foam Moisture Content (DRY Avg.): 3.56% [Karl Fischer Method] Indention Force Deflection (IDF): [ASTM D3574 Test B1] IDF @ 25%: (N) 133% IDF @ 65%: (N) 346% Resiliency/Rebound Test (RT [ASTM D3574 Test B1] RT @ 25%: (N) 121% Compression Set (DRY Avg.): [ASTM D3574] 25%: 16.0% 50%: 36.0% Tensile Strength (Dry): 30.0 lbf/In2 [ASTM D3574 Test B1].

Returning reference to FIGS. 6A, 6B, and 6C, the tube 104 is illustrated. The tube 104 may include a selected length and include a first terminal end 200 which may be connected to a connector, such as a barb connector 204. The tube 104 may be any appropriate tube, as discussed herein. Generally the tube may be medical grade and/or include a composition (e.g. anti-bacterial medical grade material) for extended and non-toxic use in an oral cavity of the user. The tube 104, in various embodiments, may include TYGON® ND 100-65 tubing having selected characteristics such as having or being made from a biocompatible non-DEHP polymer material developed and available from Saint-Gobain Performance Plastics having a place of business in Akron, Ohio. The tube 104 may have further various characteristics, as discussed herein.

The barb connector 204 may fit into the terminal end 200 and allow for the tube 104 to be connected to a selected system. As discussed above, the tube 104 may be connected to a vacuum source which may include a portable pump, a fixed pump, or a fixed source of a vacuum. In various embodiments, the vacuum source may include a hospital vacuum system which may include a whole hospital vacuum, suction systems used in dental offices, etc. The tube 104, therefore, may allow for connection of the mouthpiece 12 from the selected user and to the vacuum source for withdrawing saliva from the user. The user, given the length of the tube 104, may be able to move freely and selectively even while the mouthpiece 12 is in place. It is understood, however, that the tube 104 may also be used to deliver a saliva replacement through the mouthpiece 12 to the user.

As discussed above the tube 104 may include a cut or notched region 112. The cut or notched region may include a portion, such as about one-half, of the tube 104 removed from the tube 104 over a selected distance 112d, such as about 5 millimeters (mm) to about 50 mm, including about 25 mm, from the tube 104. The tube 104 may include an external diameter or outer diameter 104od and an internal diameter 104id. The external diameter 104od may be about 2.0 mm to about 0.1 mm, including about 0.2 mm to about 0.1 mm, and further including about 0.19 mm. The internal diameter 104id may be about 1.5 mm to about 0.01 mm, including about 0.5 mm to about 0.1 mm, and further including about 0.13 mm. At the cut portion 112, a dimension 112c from the external surface to an edge of the cut wall may be about 0.1 mm. It is understood, however, that the length of the tube, the internal diameter of the tube, and the like may be selected based upon the amount of material to be removed and/or delivered through the mouthpiece 12, the vacuum force and/or flow force of the material through the tube 104, or other factors.

The tube 104 may have properties such that at the noted dimensions it will not collapse with selected pulsations. As discussed herein, the mouthpiece may be placed in a patient's mouth and a pulse of vacuum and/or fluid delivery may occur through the tube. Thus, the tube 104 will not collapse under the negative pressure within the tube 104 due to suction, especially pulsatile suction therethrough.

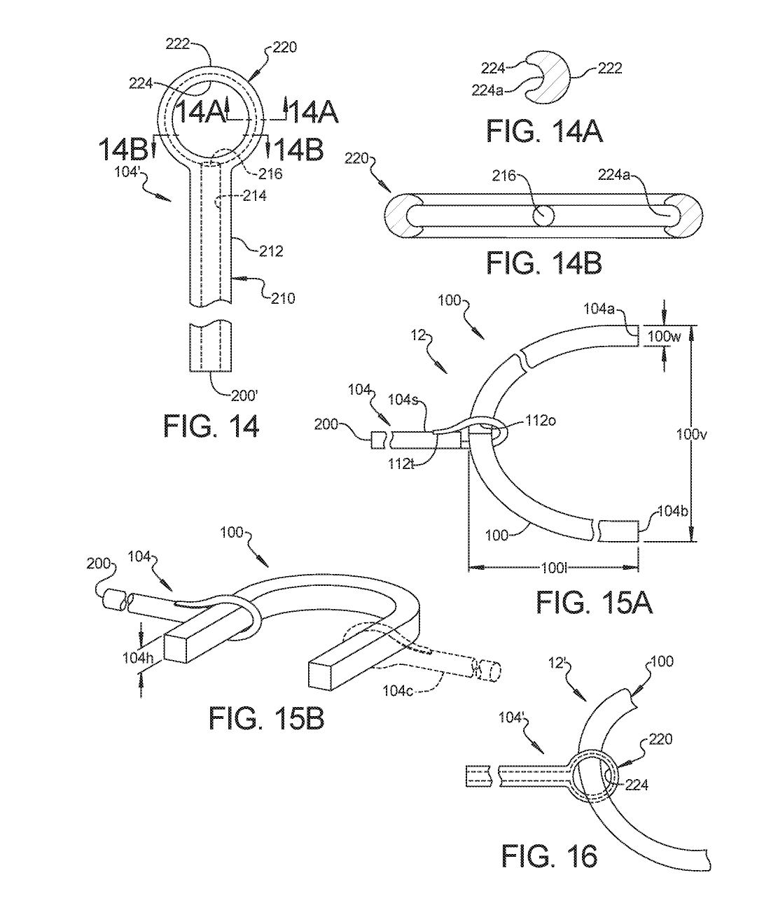

Turning reference to FIG. 14 and FIG. 14A, a tube 104′ is illustrated. The tube 104′ may be formed as a single member without a cut portion, in other words without the notch 112. The tube 104′ includes an elongated portion 210 that includes an external wall 212 and defines and internal cannula 214. The tubing 104′ may terminate or have the internal cannula 214 terminate at an opening 216. The tube 104′ may further include a terminal end 200′ similar to the end 200 of the tube 104.

Near or formed around the terminal opening 216 is a loop or integral loop or hoop, also referred to as a capture portion, 220. The hoop 220 may include an external surface or wall 222 and an internal surface or wall 224. With additional reference to FIGS. 14A and 14B, the internal surface may be formed to include a depression or groove 224a around all or a portion of the hoop 220. The groove 224a may be in communication with the terminal opening 216. Thus, a suction or fluid delivery may be made through the passage 214 and to the groove 224a. The hoop 220, including the groove 224a, may be molded to and/or with the tube 104′. The tube 104′ may be formed as one integral piece including the elongated portion 210 and the hoop 220.

In various embodiments, the tube 104′ may be similar to a sewing needle where the shaft of the needle is hollow to form the cannula 214 and the eye of the needle is the molded portion 220. The tubing 104′, however, may be formed of the similar or identical material to the tube 104. The tube 104′ may include or be formed from TYGON® ND 100-65 tubing and may be molded to include the selected hoop 220.

With continuing reference to FIGS. 6A-6C and FIG. 14, the tube 104, 104′ is connected to the foam 100 to form the mouthpiece 12, as illustrated in FIG. 15A, FIG. 15B, and FIG. 16. In FIG. 15A and FIG. 15B the tube 104, as illustrated in FIGS. 6A-6C, is formed so that the notched portion 112 is folded upon an outer wall or surface 104s of the tube 104. A terminal end portion 112t may be folded toward the terminal end 200 and placed on the outer surface 104s of the tube 104. The terminal end 112t may be fixed to the outer surface 104s in any appropriate manner, such as with an adhesive, a physical member (e.g. a zip tie or shrink tube), or other appropriate fixation. In various embodiments the terminal end 112t is adhered to the outer surface 104s with a medical grade adhesive such as 4011Prism® Medical Device Instant Adhesive, sold by Henkel Corporation having a place of business at Stamford, Conn. It is understood that any appropriate amount of the notched portion 112 may be fixed to the outer surface 104s and not simply at the immediate terminal end, but may extend a selected distance such as about 1 mm, about 2 mm, or an appropriate length from the terminal end 112t to allow for appropriate adhesion of the notched portion to the outer surface 104s of the tube 104. The foam member 100 is positioned through the loop or opening 112o. The foam 100 may be pulled through the opening 112o or the cut portion 112 may be folded over the foam member 100 during assembly and production of the mouthpiece 12.

As illustrated in FIG. 15A and FIG. 15B, the mouthpiece 12 may be formed of only the tubing 104 and the foam 100. The foam 100 may be formed of a selected material such as an open cell foam, appropriate open cell foams include an open cell foam designated FLTZ90CLMA sold by UFP Technologies, Inc. having a place of business in Grand Rapids, Mich. The foam 100 may be formed into a selected specific shape, such as generally C-shaped or semi-circular such as by di-cutting or cutting the foam material into a selected arc shape to allow it to substantially shaped, mold, or be fitted to the buccal surface 62 of the dental arch in the user and substantially along and on the gum line of the user. Accordingly, the mouthpiece 12, as noted above, may be formed from only the two portions of the tube 104 and the foam member 100. As discussed above the mouthpiece 12, may then be connected to a selected vacuum and/or salvia replacement source for treatment of the user.

The foam member 100 may include selected dimensions, as well. For example, the foam member 100 may be die cut or molded to have a selected shape. In various embodiments, the foam member 100 may have a shape that is generally a “U” and/or a “C” and/or horseshoe shaped to fit naturally in a mouth around the dental arch. The foam member 100 may have a height 100h of about 5 mm to about 20 mm, including about 11 mm. The foam member 100 may have a width 100w of about 5 mm to about 10 mm, including about 7.6 mm. The foam member 100 may have a length 1001 of about 10 mm to about 70 mm, including about 50 mm. The foam member 100 may have a span 100v of about 5 mm to about 100 mm, including about 20 mm to about 40 mm.

The tube 104 may have the opening 112o formed such that it may move along a length or portion of the foam member 100. As illustrated in FIG. 15B, the tube 104 may be moved to the position illustrated in phantom tube 104c. Thus, even during use, the user or assistant may move the tube 104 relative to the foam member 100 and/or relative to the user's mouth for various reasons. Moving the tube 104 may assist in comfort to the patient.

Moreover, it is understood that more than one tube 104 may be connected to or positioned on the foam member 100. For example, two or more tubes may be connected, such as the tube 104 and the tube 104c (shown in phantom), may be present and connected to the foam member 100 simultaneously. In this instance, the tube 104 may form suction through the foam member 100 and the tube 104c may provide an oxygen flow to the user. Thus, the mouthpiece, according to various embodiments, may include more than one tube connected to the foam member 100.

With reference to FIG. 16 the tube 104′ may also be used to form the mouthpiece 12 by pushing or moving the foam 100 through the molded loop 220, such as passing the foam 100 through the inner surface 224. The mouthpiece 12 may be formed by efficiently moving the foam member 100 through the opening 224 of the anchor or looped portion 220. The foam 100 may be substantially similar or identical to the foam 100 illustrated in FIG. 15A. Further, the tube 104′ may also be moved by the user or assistant during use of the mouthpiece, as discussed above.

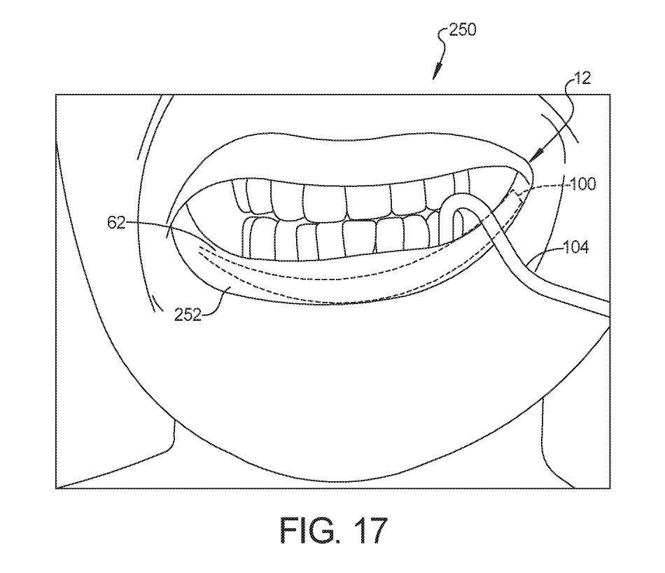

With reference to FIG. 17, the mouthpiece 12, for example the mouthpiece 12 as illustrated in FIG. 15, may be positioned relative to a user 250. The mouthpiece 12 may be positioned in the vestibule near the buccal surfaces 62 of the teeth of the user 250. The mouthpiece 12 may be positioned, in various embodiments, so that the tubing 104 may be looped or hooked over a lip 252 of the user 250. The mouthpiece 12, therefore, may be used to deliver a saliva replacement to the patient 250 and/or remove saliva from the patient 250. The mouthpiece 12, as illustrated above, may allow a vacuum to be drawn through the tubing 104, 104′, such as through the cannula of the tubing 104, 104′. The vacuum is drawn relative to the foam 100 that is positioned relative to the openings 112o and 224 of the respective tubing 104, 104′.

The foam 100 may collect saliva along the length of the foam 100 and it will be drawn through the foam 100 including a substantially open cell configuration and through the respective tubing 104, 104′. The open cell foam of the foam 100 allows a vacuum to be drawn at substantially only a single location (i.e. the location of the tube 104) and a vacuum to drawn liquid throughout the foam member 100 into the tube 104.

Thus, the foam 100 allows a vacuum to be drawn within a large area with the mouth of the user.

Accordingly the foam 100 need not, as illustrated in FIG. 15 and FIG. 16, be encapsulated or covered entirely by any other material or member. As discussed herein and illustrated a passage or loop portion of the tube 104, 104′ may encompass at most or only a portion of the foam member 100. The tube 104, 104′ may cover a selected length of the foam member 100, such as generally the outer diameter of the tube 104, 104′. Accordingly the tubing 104, 104′ may cover about 0.5% to about 10%, including about 0.5% to about 3%, and further including about 0.5% to about 1%, of a length from a first terminal end 100a to a second terminal end 100b of the foam 100. Thus, the mouthpiece 12 may be substantially worn by the user 12 as the open cell foam 100 is open to the mouth of the user 250.

In various embodiments, the tube 104, 104′ may be moved relative to the foam member 100 as well and/or in addition to movement relative to the patient. The openings 112o, 224 may allow the end of the respective tubes 104, 104′ to be moved relative to the foam member 100. For example, the opening 112o, 224 may be initially positioned at or near a selected end of the foam member 100. After a selected period of time, such as 60 minutes, the opening 112o, 224 may be moved to a second position such as near a second end of the foam member 100. Thus, the tube end opening 112o, 224 need not be fixed at a location or position relative to the foam member 100.

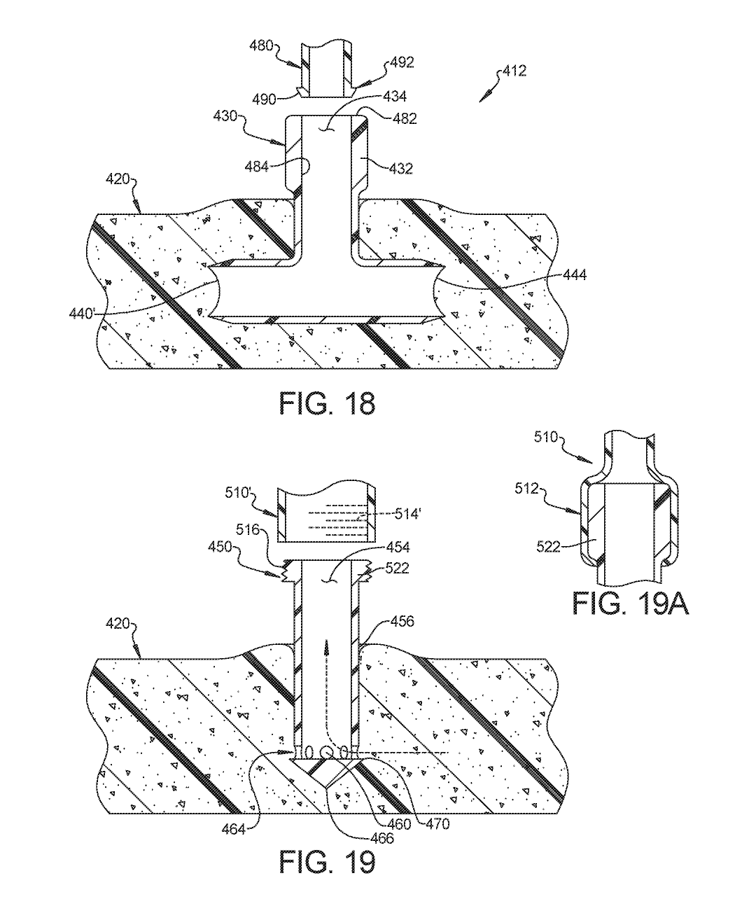

With reference to FIG. 18, a mouthpiece 412 is illustrated. The mouthpiece 412 may include portions that are similar to those discussed above, including a mouth member or foam member 420. The foam or mouth member 420 may be similar to the foam member 100, as discussed above. Generally, the foam member 420 may be formed of an appropriate shape, such as a U or C shape, to be positioned within the mouth of a user or patient, as illustrated in FIG. 17. Further the foam member 420 may be formed of material similar to that discussed above.

The foam member 420 may be an open cell foam which may allow it to be pierced or formed around a nozzle or connection member 430. The nozzle or connection member 430 may be formed to be positioned within the foam member 420 such as by pushing, molding, or the like. The connection member 430 includes an external wall 432 that defines or forms an internal passage 434 by an internal surface 484. The connection member 430 may be formed of a selected rigid or substantially rigid material to allow the connection member 430 to be press fit into the foam 420.

The internal passage 434 may pass through the external wall 432 into one or more openings, such as a first opening 440 and a second opening 444. The two openings 440 and 444 may be within the foam member 420. Accordingly, a suction or vacuum may be drawn through the opening of passage 434, and also therefore through the openings 440 and 444, relative to the foam member 420. When a vacuum is drawn, a vacuum or suction may be formed through the foam member 420 through the connection member 430. It is understood that the connection member 430 may be formed with any appropriate number of openings within the foam member 420 and may be fit into the foam member 420 in an appropriate manner.

Turning reference to FIG. 19, a connection member 450 may also define an internal passage 454 though an external wall 456 of the connection member 450. The external wall 456 may have one or more ports or throughbores 460 formed therethrough. Accordingly, a suction drawn through the passage or opening 454 may be drawn through the throughbores 460 such that the foam member 420 may have a vacuum drawn therein.

The connection member 450 may further include a barb or reverse angle region 464 that includes a point or projection portion 466 and one or more steps or anchor regions 470. The connection member 450 may be pushed into the foam member 420, such as after forming the foam member 420. The anchor portion or region 464 the assistant holding the connection member 450 thereto.

In various embodiments, a connection member, such as the connection member 450, may have a portion that extends into the foam member 420a selected distance. The connection member may be pushed into, pierce, and/or be formed into at least a portion of the foam member 420. The passages, such as the passages 460, however, are within the foam member 420 and may provide a fluid thereto or have a suction drawn therethrough, as discussed above. Thus, the connection member, such as the connection member 450, may extend from more than one side of the foam member 420.

With continuing reference to FIG. 18 and FIG. 19, and additional reference to FIG. 19A, a tube or connection portion 480 may connect with the connection member 430 in a selected manner. Near a top or first end 482 of the connection member 430 the opening 434 may be defined by the internal wall 484. The internal wall 484 may include a taper or straight wall portion that engages a taper or connection taper 490 formed at an end 492 of the tube 480. The tube 480 may be similar to the tube 104, as discussed above, and it may be connected with a selected vacuum source or fluid source, such as a pump, for drawing a vacuum through the connection member 430 and the foam member 420, respectively.

The tapered portion 490 may allow for a sealed connection with the connection member 430. The tapered portion 490 may allow for an easy and efficient connection of the tube member 480 with the connection member 430 such as by a press fit of the tube member 480 into the opening 434 ending contact with the internal wall 484. Thus, the tube member 480 might be connected with the connection member 430 in a removable manner. However, during extended or selected periods of use, the tapered portion 490 is maintained connected with the connection member 430. It is also understood by one skilled in the art, additional materials such as adhesives or sealants may be added to assist in creating or maintaining a seal and/or maintaining a connection of the tube 480 with the connection member 430. Adhesives or sealants may be used as an alternative to the tapered portion 490 as well.

In various embodiments, with reference to FIG. 19 and FIG. 19A, a tube 510 may be formed or have a portion, such as near a distal end 512 that is substantially elastic or elastically deformable. The connection member 450 may include a barb or connection portion, such as an upper rim 522 over which the elastic portion 512 may be expanded and then elastically connects thereto. According, the tube 510 may be elastically deformed over the connection or rib portion 522 and then held thereto, such as with a friction fit or elastic connection during use of the foam member 420 as the mouthpiece, similar to that as discussed above. Accordingly, the tube 510 may be selectively connected to the connection member 450 during use or for use of the foam member 420 in a suction and/or fluid may be supplied through the tube member 510 to the connection member 450.

A tube 510′ may also or alternatively include a thread 514. The thread 514 may be an internal thread that is configured to engage an external thread 516 of the connection member 450. The threads 54, 516 may be reversed such that the thread 514 of the tube is external and the thread 516 of the connection member is internal. Nevertheless, the tube 510′ may be threadably connected to the connection member 450.

Accordingly, with reference to FIGS. 18-19A, tube members may be selectively connected to the connection members in an appropriate manner, as discussed above. Further, various features or elements may be selectively positioned, such as the elastic portion 512 interconnecting with the connection member 430 rather than the tapered portion. It is further understood that various elements, as discussed above, may be interchanged in various embodiments unless specifically identified to the contrary.

It is further understood that the mouthpiece, according to various embodiments, may be provided and used for appropriate applications in selected subjects, such as human patients. Thus, a gas, such as air or oxygen gas, may be passed to and through the mouthpiece to the user. Also, the mouth piece may be used with a pump system that may be used to both deliver a fluid and draw a vacuum with the same unit and as selected. Further, a selected agent, such as a sour tasting agent, may be delivered periodically through the mouthpiece to the the patient to enhance to help with the production of saliva. Thus, even with a vacuum, the patient may not experience an overly dry mouth and saliva production may be enhanced.

It is further understood that the tube member of the mouthpiece may be appropriately sized to operate with the foam member, such as the foam member 100. The tube 104 may have the end or opening 112o that is sized to allow or form a suction or vacuum in the foam member 100 when a suction is present in the tube 104. Thus, the foam may have an exterior dimension that is the same or larger than the opening 112o and/or the foam member 100 may be in contact with the interior 224 of the tube 104′. The foam member 100 may also be smaller than the openings and/or not be in total contact with the internal surfaces if a suction is formed in the foam member 100 and/or fluid is sufficiently provided thereto.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A system for providing fluid to and/or removing fluid from a mouth cavity of a user, the mouth cavity having a dental arch with a biting surface and vestibule on an outer side of the dental arch, and a gum line on an inner side of the dental arch, the system comprising:
a mouthpiece having only (i) a hydrophilic foam member operable to shape to the dental arch, (ii) a portion of a conduit in fluid communication with the hydrophilic foam member; and
a fluid conduit extension member coupled to the portion of the conduit.

2. The system of claim 1, wherein the hydrophilic foam is an open cell foam.

3. The system of claim 1, wherein the hydrophilic foam is formed into a substantially U-shape to fit within the mouth of the user along the gum line.

4. The system of claim 1, wherein the hydrophilic foam includes an open cell foam wherein a vacuum drawn at substantially one location of the hydrophilic foam member is drawn throughout the hydrophilic foam member.

5. The system of claim 1, further comprising:
a supply source of a vacuum coupled to the fluid conduit extension member.

6. The system of claim 5, wherein the vacuum source comprises a pump and a controller configured to regulate the flow of saliva through the fluid conduit.

7. The system of claim 5, wherein the supply source of the vacuum is portable.

8. The system of claim 1, further comprising:
a supply source of a fluid coupled to the fluid conduit extension member.

9. The system according to claim 1, wherein the portion of the conduit includes:
a terminal end that is enclosed and encompasses a portion of the hydrophilic foam member therein; and
a passage in fluid communication with the hydrophilic foam member.

10. The system according to claim 1, wherein the portion of the conduit includes:
a terminal end wall portion that encompasses at least a portion of hydrophilic foam member; and
a passage in fluid communication with the hydrophilic foam member.

11. The system of claim 1, further comprising:
a connection member connected to the hydrophilic foam member;
wherein the fluid conduit extension member is connected to the connection member.

12. The system of claim 11, wherein the connection member is pressfit into the hydrophilic foam member.

13. The system of claim 11, wherein the fluid conduit extension member is coupled to the connection member via a pressfit, elastic coupling, threaded coupling, adhesive, or combinations thereof.

14. A method for providing fluid to and/or removing fluid from a mouth cavity of a user, comprising:
positioning a mouthpiece having only (i) a hydrophilic foam member operable to shape to the dental arch and (ii) a portion of a conduit in fluid communication with the hydrophilic foam member in a mouth cavity having a dental arch with a biting surface and vestibule on an outer side of the dental arch, and a gum line on an inner side of the dental arch; and
coupling a fluid conduit extension member to the portion of the conduit.

15. The method of claim 11, further comprising:
forming the hydrophilic foam member of an open cell foam.

16. The method of claim 11, further comprising:
cutting a terminal end of the portion of the conduit;
folding the cut terminal end over the hydrophilic foam member; and
exposing the hydrophilic foam member to a fluid passage through the portion of the conduit.

17. The method of claim 14, further comprising:
adhering the cut terminal end to an exterior surface of the portion of the conduit to encompass at least a portion of the hydrophilic foam member.

18. The method of claim 11, further comprising:
providing a terminal end of the portion of the conduit to have an enclosed passage;
positioning the hydrophilic foam member within the enclosed passage; and
exposing the hydrophilic foam member to a fluid passage through the portion of the conduit.

19. The method of claim 14, further comprising:
coupling the portion of the conduit to a connection member that is coupled to the hydrophilic foam member.

20. A system for providing fluid to and/or removing fluid from a mouth cavity of a user, consisting essentially of:
a mouthpiece having (i) having a hydrophilic foam member operable to shape to the dental arch and (ii) a portion of a conduit in fluid communication with the hydrophilic foam member; and
a fluid conduit extension member coupled to the portion of the conduit.
